(19) United States
(12) Patent Application Publication
(10) Pub. No.: US 2025/0214053 A1
(43) Pub. Date: Jul. 3, 2025

(54) METHOD AND DEVICE FOR SYNTHESIZING MOLECULES

(71) Applicant: CYTIVA SWEDEN AB, Uppsala (SE)

(72) Inventors: Per Denker, Uppsala (SE); Niklas Edblad, Uppsala (SE); Johan Arthursson, Uppsala (SE); Erik Estrada, Uppsala (SE); Ulrika Skarp, Uppsala (SE); Magnus Råberg, Uppsala (SE); Mats Hansson, Uppsala (SE); Daniel Öman, Uppsala (SE); Stanislaw Zabramski, Uppsala (SE); Eva Isacsson, Uppsala (SE); Hans Axelsson-Svala, Uppsala (SE); Mats Litgård-Maot, Uppsala (SE); Lennie Fredriksson, Uppsala (SE); Petrus Lindberg Borner, Uppsala (SE)

(21) Appl. No.: 18/841,907

(22) PCT Filed: Feb. 23, 2023
(86) PCT No.: PCT/EP2023/054614

§ 371 Date: Aug. 27, 2024

(30) Foreign Application Priority Data
Mar. 3, 2022 (SE) 2250288-4

(51) Int. Cl. B01J 19/00; G16C 20/10

(52) U.S. Cl. CPC B01J 19/0046; G16C 20/10; B01J 2219/00689; B01J 2219/00695; B01J 2219/00722

(57) ABSTRACT

The present invention relates to a computer implemented method performed by a device (600) configured to synthesize molecules, wherein the synthesizing comprises performing cycles of a plurality of process steps (PS1-PS4), wherein the synthesizing is controlled by blocks of parameters, the method comprising receiving an indication of a target molecule, generating a synthesizing process description (300) by generating a plurality of blocks (310) of parameters, each block (310) controlling process steps of a respective cycle, receiving user input indicative of modification of the generated plurality of blocks of parameters, modifying the generated plurality of blocks of parameters using the user input, controlling synthesizing of the target molecule by applying the modified plurality of blocks of parameters to control of the respective cycles.

### TECHNICAL FIELD

The present invention relates to a method for synthesizing molecules. The invention further relates to a device configured to synthesize molecules.

### BACKGROUND

Synthesizing of molecules are commonly used in, for example, production of diagnostic kits, clustered regularly interspaced short palindromic repeats (CRISPR) treatment and oligonucleotide therapeutics. Examples of diagnostic kits are kits for testing for COVID-19. One example of oligonucleotide therapeutics is e.g., Spinraza® (Nusinersen) for treating infants diagnosed with Spinal Muscular Atrophy. Another example oligonucleotide therapeutics is Leqvio® (Inclisiran) treatment for high cholesterol.

Examples of synthesized molecules are oligonucleotides and oligopeptides.

An example of a conventional method for synthesizing molecules are described in US20200032250A1.

However, a drawback with such conventional methods is that they are not flexible in terms of synthesis methods, synthesis chemistry and synthesis scale. Another drawback is that efficiency in the use of reagents is not as good as one would desire.

Thus, there is a need for an improved method for synthesizing molecules.

### Objects of the Invention

An objective of embodiments of the present invention is to provide a solution which mitigates or solves the drawbacks and problems described above.

### SUMMARY OF THE INVENTION

The above and further objectives are achieved by the subject matter described herein. Further advantageous implementation forms of the invention are further defined herein According to a first aspect of the invention, the above mentioned and other objectives are achieved by a computer implemented method performed by a device configured to synthesize molecules, wherein the synthesizing comprises performing cycles of a plurality of process steps, wherein the synthesizing is controlled by blocks of parameters, the method comprising receiving an indication of a target molecule, generating a synthesizing process description by generating a plurality of blocks of parameters, each block controlling process steps of a respective cycle, receiving user input indicative of modification of the generated plurality of blocks of parameters, modifying the generated plurality of blocks of parameters using the user input, controlling synthesizing of the target molecule by applying the modified plurality of blocks of parameters to control of the respective cycles.

At least one advantage of the disclosure according to the first aspect is an improved process for synthesizing molecules is achieved by in a flexible manner optimizing cycles as well as process steps.

In one embodiment according to the first aspect, each cycle comprises a plurality of process steps and wherein each respective block comprises a corresponding number of sub-blocks of parameters as the number of process steps in the plurality of process steps, wherein the received user input is further indicative of one or more modifications of the sub-blocks, wherein controlling synthesizing further comprises applying the modified sub-blocks to control the process steps of the respective cycles.

In one embodiment according to the first aspect, modifying the generated plurality of blocks comprises applying the user input globally to all cycles by modifying respective blocks of parameters.

In one embodiment according to the first aspect, modifying the generated plurality of blocks comprises applying the user input to all cycles of the same type by modifying respective blocks of parameters.

In one embodiment according to the first aspect, the user input is further indicative of selected cycles of the process description, wherein modifying the generated plurality of blocks comprises applying the user input to the selected cycles.

In one embodiment according to the first aspect, the target molecule is an oligonucleotide, and the plurality of process steps at least includes detritylation, coupling, oxidation and capping.

In one embodiment according to the first aspect, the respective sub-bloc for the process step detritylation comprises parameter values for start level, end level, linear flow, efficiency limit, wash CV, wherein the respective sub-bloc for the process step coupling comprises parameter values for base identity, Eq, Amidite concentration, coupling CT, percentage activator, push flow, recycle time and wash CV, wherein the respective sub-bloc for the process step oxidation comprises parameter values for ox CT DNA, ox Eq DNA, ox CT RNA, ox Eq RNA, chase CV ox, wash CV ox, wherein the respective sub-bloc for the process step capping comprises parameter values for cap CT, cap CV, chase CV ox and wash CV ox.

In one embodiment according to the first aspect, the target molecule is an oligopeptide, and the plurality of process steps at least includes detritylation, coupling and optionally capping.

In one embodiment according to the first aspect, receiving user input comprises: rendering a representation of the synthesizing process description, wherein the representation comprises representations of blocks and/or sub-blocks and/or parameter values, displaying the representation to a user, receiving user input indicative of modification of the generated plurality of blocks of parameters by receiving user input indicative of interaction with the representation.

In one embodiment according to the first aspect, the interaction is at least indicative of modified parameter values and indicative of that the modified parameter values should be applied globally to all cycles by modifying respective blocks of parameters.

In one embodiment according to the first aspect, the interaction is at least indicative of modified parameter values and indicative of that the modified parameter values should be applied to all cycles of the same type by modifying respective blocks of parameters.

In one embodiment according to the first aspect, the interaction is at least indicative of selected cycles of the process description and indicative of modified parameter values and indicative of that the modified parameter values should be applied to the selected cycles.

According to a second aspect of the invention, the above mentioned and other objectives are achieved by a device configured to synthesize molecules, wherein the synthesizing comprises performing cycles of a plurality of process steps, wherein the synthesizing is controlled by blocks of parameters, the device comprising: a controllable fluid network, an input device, a display, and a control unit comprising circuitry, the circuitry comprising: a processing circuitry, and a memory, said memory comprising instructions executable by said processing circuitry, wherein the control unit is communicatively coupled to controllable units of the fluid network, whereby said device is configured to perform the method according to the first aspect when the instructions are executed by said processing circuitry.

According to a third aspect of the invention, the above mentioned and other objectives are achieved by a computer program comprising computer-executable instructions for causing a control unit, when the computer-executable instructions are executed on processing circuitry comprised in the control unit, to perform the method according to the first aspect.

According to a fourth aspect of the invention, the above mentioned and other objectives are achieved by a computer program product comprising a computer-readable storage medium, the computer-readable storage medium having the computer program according to the third aspect embodied therein.

A more complete understanding of embodiments of the invention will be afforded to those skilled in the art, as well as a realization of additional advantages thereof, by a consideration of the following detailed description of one or more embodiments. It should be appreciated that like reference numerals are used to identify like elements illustrated in one or more of the figures.

### BRIEF DESCRIPTION OF THE DRAWINGS

FIG. 1 illustrates a process cycle for synthesizing a molecule and is comprising a plurality of process steps according to one or more embodiments of the present disclosure.

FIG. 2 illustrates a process cycle for synthesizing an oligonucleotide according to one or more embodiments of the present disclosure.

FIG. 3 illustrates an example of a process description configured to synthesize a molecule according to one or more embodiments of the present disclosure.

FIG. 4 illustrates selected blocks of parameters according to one or more embodiments of the present disclosure.

FIG. 5 illustrates a computer implemented method performed by a device configured to synthesize molecules according to one or more embodiments of the present disclosure.

FIG. 6 illustrates a device configured to synthesize molecules according to one or more embodiments of the present disclosure.

FIG. 7 illustrates an example of a device configured to synthesize oligonucleotides according to one or more embodiments of the present disclosure.

FIG. 8 illustrates an example of a device configured to synthesize oligonucleotides according to the present disclosure.

A more complete understanding of embodiments of the invention will be afforded to those skilled in the art, as well as a realization of additional advantages thereof, by a consideration of the following detailed description of one or more embodiments. It should be appreciated that like reference numerals are used to identify like elements illustrated in one or more of the figures.

### DETAILED DESCRIPTION

Related patents/applications EP0844910B1, GB 2 118 139 and U.S. Pat. No. 4,589,049 are included by reference herein.

An “or” in this description and the corresponding claims is to be understood as a mathematical OR which covers “and” and “or”, and is not to be understand as an XOR (exclusive OR). The indefinite article “a” in this disclosure and claims is not limited to “one” and can also be understood as “one or more”, i.e., plural.

In the present disclosure reference will be made interchangeably to processing circuitry and processing means.

In the present disclosure the term “fluid source” denotes a unit or arrangement capable of providing a fluid, e.g., a fluid container, a fluid tank or a fluid reservoir. In the following description the fluid source will be described as a container, but any suitable fluid source may be used.

In the present disclosure the term “block of parameters” denotes a set of parameters, where each parameter is assigned a parameter value. The set of parameters controls the behavior or characteristics of a device configured to synthesize molecules in a particular process cycle, where the process cycle comprises a plurality of process steps. The block of parameters may comprise or be divided into sub-blocks of parameters, where each sub-block controls the behavior or characteristics of the device when performing a particular process step. It is understood that a plurality of subsequent blocks can control a plurality of subsequent cycles, where each cycle of the plurality of subsequent cycles comprises a plurality of process steps.

In the present disclosure the term “synthesize molecules” denotes performing a plurality of cycles of process steps, thereby adding molecule components to a chain of molecule components to form a target molecule. One example of synthesizing molecules is generating oligonucleotide chains from support-bound nucleosides. One further example of synthesizing molecules is generating oligopeptide chains from amide bound amino acids.

In the present disclosure the term “target molecule” denotes a desired molecule formed/generated by performing cycles of process steps and adding components until a desired chain of components forms the target molecule. Example molecules are oligonucleotides or oligopeptides.

In the present disclosure the term “controlling synthesizing” denotes controlling a device configured to synthesize molecules.

In the present disclosure the term “a fluid network” denotes fluidly coupled device components configured to hold fluids, direct fluids and control flow of fluids. Examples of device components comprise in a fluid network are conduits/pipes, fluid containers/receptacles, sensors, valves, fluid columns etc. Some device components may be controllable and are communicatively coupled to a control unit, and reacts to control signals received from the control unit and/or send data, e.g., device component characteristics and/or measurement data, as control signals to the control unit. Examples of such controllable device components are sensors and valves.

The present disclosure relates to an improved method and device for synthesizing of molecules. The molecules are typically oligonucleotides and oligopeptides.

An oligonucleotide is a macromolecule comprising a sequence of nucleosides, each of which includes a sugar and a base. Each nucleoside is separated from adjacent nucleosides with an internucleotide linkage, which effectively serves to bond the nucleosides together. The sugar is generally a pentose, most commonly a deoxyribose, ribose, or 2′-0-substituted ribose. A number of different bases can be used, the four most common of which are adenine, cytosine, guanine, and thymine (abbreviated as A, C, G, and T, respectively). The internucleoside linkage is most commonly a phosphate, which may be substituted with a variety of substituents at a nonbridging oxygen atom, most commonly by sulphur or an alkyl, ester, or amide group.

Different methods are used for synthesizing oligonucleotides, including phosphoramidite, phosphotriester, and H-phosphonate methods, each of which is generally known in the field of molecular biology. The phosphoramidite method is described here as an exemplary method. To produce a large number of oligonucleotide molecules with this method, a solid support is provided in a reaction vessel and a large number of DMT-protected nucleosides are fixed to the support.

In a removal or detritylation process step, a deprotectant, e.g., acting through a detritylation mechanism, is added to remove the DMT from nucleoside, and thus to “deprotect” that one hydroxyl. As a result, the last nucleoside in the sequence has one hydroxyl that is ready to receive a next amidite.

In a coupling process step, a nucleoside phosphoramidites (hereafter “amidites”), dissolved in a solvent such as acetonitrile (ACN), are introduced into the vessel. An activator, such as tetrazole, is also introduced into the vessel with the amidites. The phosphorus in the amidites bonds with the oxygen in the hydroxyl, thus providing support-bound nucleotides. After the support-bound nucleotides are formed, excess amidites are flushed from the vessel with ACN.

In an oxidation process step, an oxidizing agent is added to convert the trivalent phosphorous to pentavalent.

In a capping process step, a capping agent is added to block all the unprotected hydroxyls from reacting with amidites introduced at a later stage. ACN is again introduced to flush out the capping agent.

These process steps are repeated/cycled any number of times to produce growing, oligonucleotide chains from support-bound nucleosides. Each of the chains should have an identical repeating sequence of nucleosides.

The process above for producing oligonucleotides is time consuming and the materials that are used, particularly the amidites, are expensive and require special handling and disposal after being used.

At least one advantage of the present disclosure is that cycles as well as process steps may be modified to optimize the process and the use of fluids, such as amidites. The modification may be performed on different levels, e.g., be performed globally, for cycles of a particular type and for individual selected cycles.

In laboratories, oligonucleotides are typically synthesized on a scale of about one micromole. One group of devices/machines used for synthesizing molecules are produced under the name OligoPilot (a trademark of the assignee of the present invention) and has improved the process to produce as much as 3-4 millimoles of molecules. It would be desirable to increase the number of molecules that can be produced at one time, and to do so efficiently with the minimal amount of reagents needed.

An oligopeptide is a macromolecule comprising a sequence of amino acids linked via amide bonds. Cycles for forming the target molecule or target oligopeptide may include the following process steps:

In a removal or Fmoc removal process step, a deprotectant, acting through a removal mechanism, is added to “deprotect” the molecule. As a result, the last amino acid in the sequence is ready to receive a next amino acid.

In a coupling process step, the chain of amino acids forms an amide bond with the next amino acid.

In an optional capping process step, a capping agent is added to block reaction with amidites introduced at a later stage.

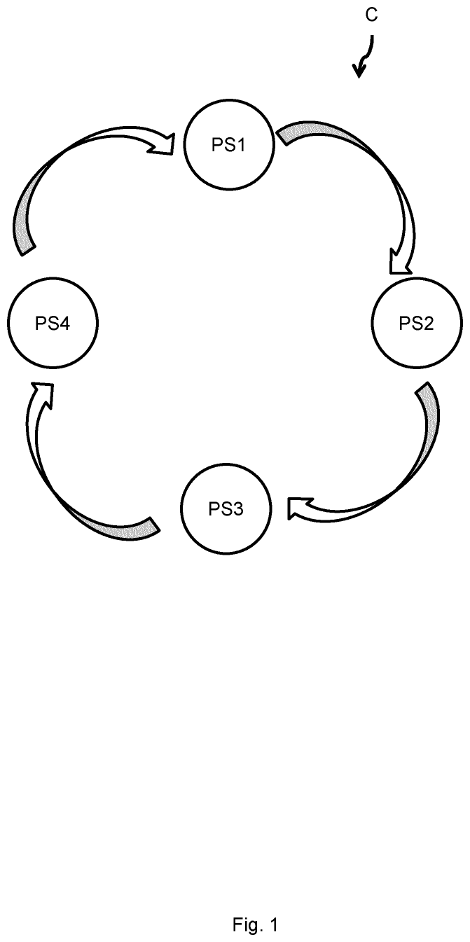

FIG. 1 illustrates a process cycle C for synthesizing a molecule and is comprising a plurality of process steps PS1-PS4 according to one or more embodiments of the present disclosure. The process steps are performed subsequently from a first process step PS1 to a last process step PS4. The cycle C may be repeated an arbitrary number of times by repeating the plurality of process steps PS1-PS4. It is understood that any number of process steps may be used without departing from the present disclosure.

In FIG. 1 four process steps are shown, however a cycle may comprise 2, 3, 5 or any greater number of process steps than that.

An additional cycle C′ (not shown) may be generated by modifying the plurality of process steps PS1-PS4. Each cycle typically adds to the chain and eventually forms a target molecule.

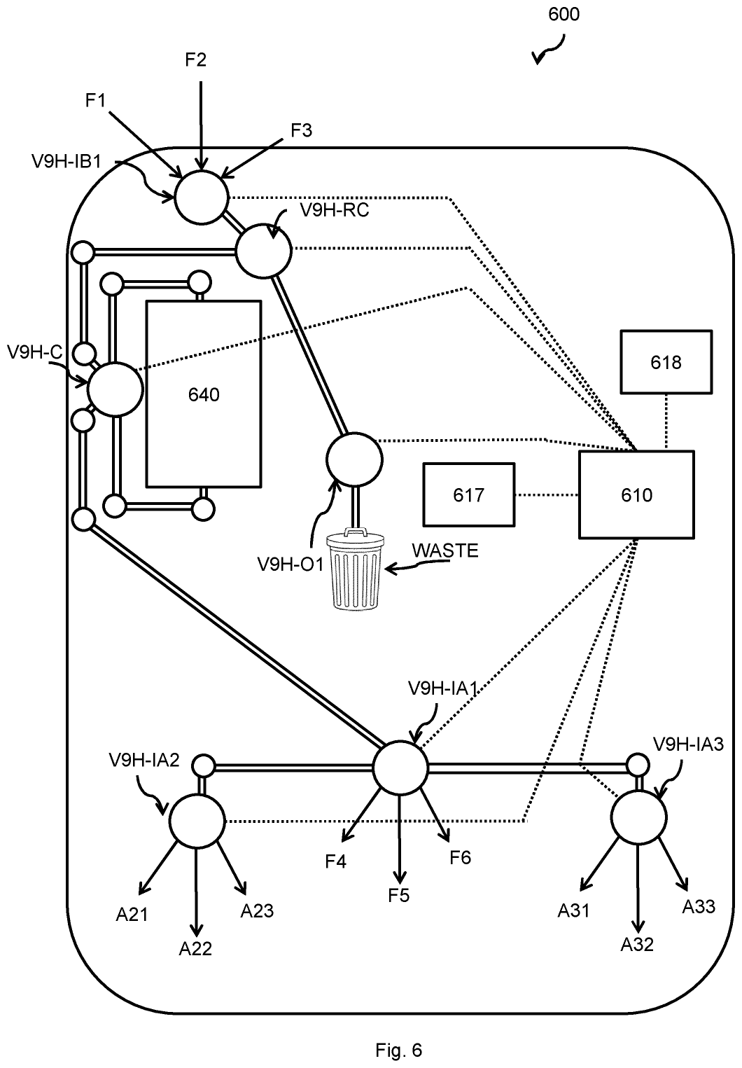

A process cycle is typically controlled by a block of parameters when the block of parameters is fed or sent to a device configured to synthesize the molecule. Devices configured to synthesize the molecule are further described in relation to FIG. 6-8. The block of parameters may be split into or comprise sub-blocks, where each sub-block is controlling a respective process step PS1-PS4.

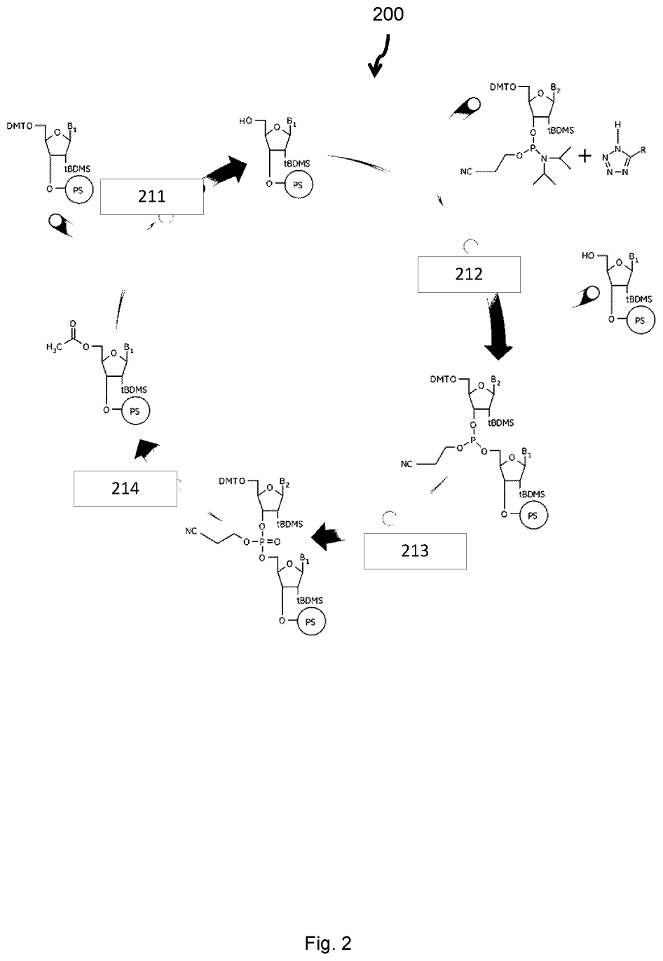

FIG. 2 illustrates a process cycle 200 for synthesizing an oligonucleotide according to one or more embodiments of the present disclosure. The process cycle 200 comprises a plurality of process steps at least including detritylation 211, coupling 212, oxidation 213 and capping 214. The process steps are further described above in the initial part of the detailed description.

In a detritylation process step 211, a “deprotectant” a DMT from nucleoside is added to “deprotect” the one hydroxyl. I.e., the last nucleoside in the sequence has one hydroxyl that is ready to receive a next amidite.

In a coupling process step 212, amidites are introduced together with an activator. Thereby support-bound nucleotides are provided.

In an oxidation process step 213, an oxidizing agent is added to convert the trivalent phosphorous to pentavalent.

In a capping process step 214, a capping agent is added to block all the unprotected hydroxyls from reacting with amidites introduced at a later stage.

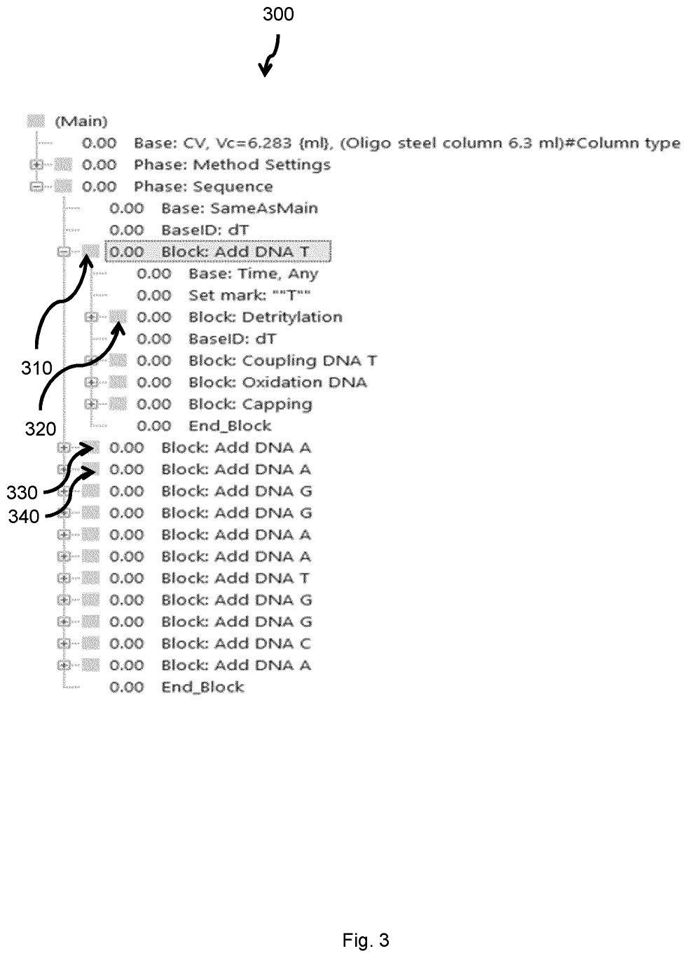

FIG. 3 illustrates an example of a process description 300 configured to synthesize a molecule according to one or more embodiments of the present disclosure. A process description configured to synthesize a molecule is commonly also referred to as a “method” in prior art. The process description 300 is indicative of parameters controlling a plurality of cycles C and respective process steps PS1-PS4. In the example shown in FIG. 3, the process description 300 is rendered as a graphical representation in a tree-like or hierarchical format. Other suitable graphical representations are also envisioned, e.g., a simple list, other hierarchical or relational diagrams such as mind maps etc. In other words, graphical representations that are capable of illustrating an order of cycles and the hierarchical relation cycle->process steps->parameters.

The process description may have a top level (Main) representing the top level of the tree/hierarchy. On a level just below the top level, the process description 300 comprises a block level, where blocks of parameters 310 controlling a respective cycle are arranged in a particular order. In FIG. 3 blocks for respective cycles are shown as “Add DNA T”, Add DNA A”, Add DNA G” etc. On a level just below the block level, the process description 300 comprises a sub-block level, where sub-blocks of parameters 310 each controlling a respective process step are arranged in a particular order, e.g., in the order shown in relation to FIG. 2. In FIG. 3 sub-blocks for respective cycles are shown as “Block: Detritylation”, “Block: Coupling DNA T”, “Block: Oxidation DNA #”, “Block: Capping” etc.

Each block of parameters may be associated with a particular type, e.g., “A”, “C”, “G” or “T”.

In one example, each of the blocks comprises a “coupling” process step. The parameters of this process step may be edited and set globally to all cycles “A”, “C”, “G” or “T” by modifying respective sub-blocks of parameters relating to the “coupling” process step. This is referred herein as global modification of parameters. Examples of globally modified parameters are “Flow rate”, “Volume”, “Chase Volume”, “Wash volume”, which in practical terms are modified to achieve optimal or maximum coupling efficiency with as little excess of regent as possible.

In one example, all blocks of parameters of type “Aϵ are modified. The parameters of this process step may be edited and set for all cycles of type “A by modifying respective parameters and/or sub-blocks of parameters. This is referred herein as modification of parameters to cycles of the same type. A first and second block of parameters 330, 340 are shown in FIG. 3. Both the first and second block of parameters 330, 340 are of the block/cycle type “A”. Examples of parameters for cycles of the same type are “Base”, “Amidite equivalents”, “Amidite concentration”, “Coupling change time”, “% Activator”, “Recirculation time”, “Coupling wash”, which in practical terms are modified to achieve optimal or maximum coupling efficiency with as little excess of regent as possible practical terms are modified to achieve optimal or maximum coupling efficiency with as little excess of regent as possible.

In one example, only selected blocks of parameters are modified. The parameters of blocks of parameters indicated by user input are modified by modifying respective parameters and/or sub-blocks of parameters. This is referred herein as modification of selected cycles/blocks of parameters of the process description 300. Examples of parameters for selected cycles are “Index”, “Base”, “Amidite equivalents”, “Amidite concentration”, “Charge time”, “Activator mix”, “Recirculation time”, “Wash volume”, which in practical terms are modified to achieve optimal or maximum coupling efficiency with as little excess of regent as possible.

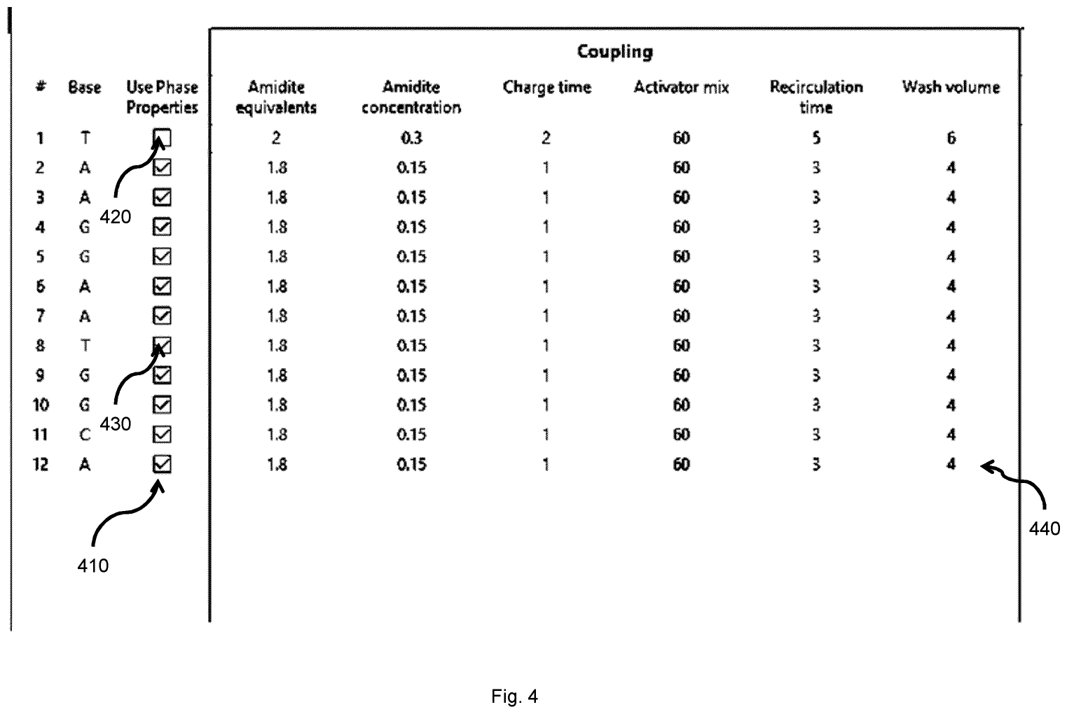

Selected blocks of parameters are illustrated in relation to FIG. 4.

FIG. 4 illustrates selected blocks of parameters 410 according to one or more embodiments of the present disclosure. Parameters 440 of a sub-block/process step “coupling” are shown to the right of the figure. Examples of parameters for selected cycles are “Index”, “Base”, “Amidite equivalents”, “Amidite concentration”, “Charge time”, “Activator mix”, “Recirculation time”, “Wash volume”.

To the left checkboxes 410 of respective cycles/blocks are ticked 430 or not ticked 420, thereby indicating a selection. I.e., a ticked box indicates a selected cycle/block. In the example shown in FIG. 4, a first cycle/block 420 of type “T” is not selected, whereas a second cycle/block 430 of type “T” is selected and will be modified using the user input.

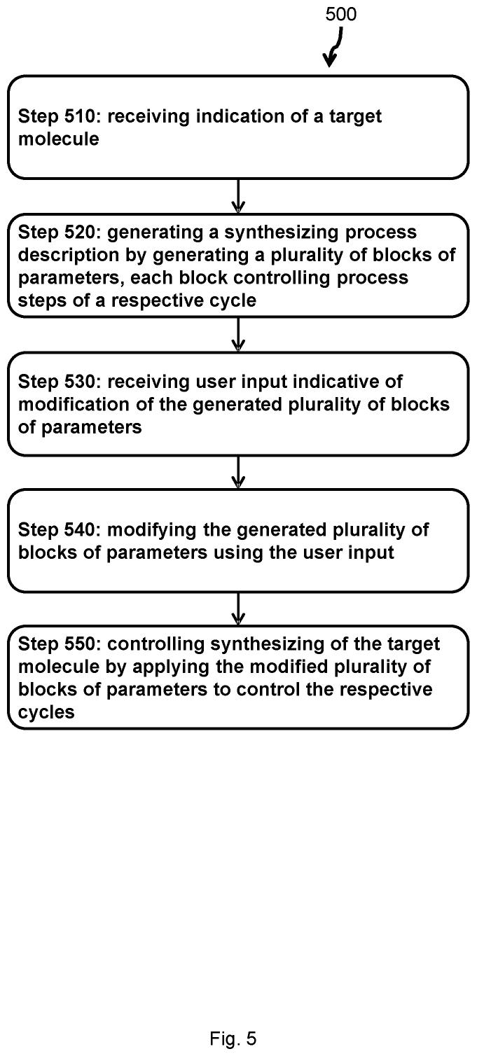

FIG. 5 illustrates a computer implemented method performed by a device configured to synthesize molecules according to one or more embodiments of the present disclosure. Synthesizing comprises performing cycles of a plurality of process steps PS1-PS4, where the synthesizing is controlled by blocks of parameters. The method comprises:
Step 510: receiving an indication of a target molecule.

In one example, a text input field is rendered and displayed to a user. The user then provides user input, for example by typing in a text string, indicative of a target molecule, e.g., “ACG GTA AAT T”. This indication is then received by the device.
Step 520: generating a synthesizing process description 300 by generating a plurality of blocks 310 of parameters, each block 310 controlling process steps of a respective cycle.

In one example, blocks of parameters are generated by performing a lookup, using the text string, in a memory or database to find blocks of known combination/s of cycles that when performed subsequently will generate the target molecule.
Step 530: receiving user input indicative of modification of the generated plurality of blocks of parameters.

In one example, the generated process description 300 is rendered and displayed in a similar manner to what is shown in relation to FIG. 3. The user may then interact with the rendered and displayed representation of the generated process description 300, e.g., by adding blocks or sub-blocks, deleting blocks and/or sub-blocks or by modifying parameter values of parameters comprised by the blocks and/or sub-blocks.
Step 540: modifying the generated plurality of blocks of parameters using the user input.

In one example, blocks and/or sub-blocks indicated by the user input are added and/or deleted. In one further example, parameters are updated with new values using the user input.
Step 550: controlling synthesizing of the target molecule by applying the modified plurality of blocks of parameters to control the respective cycles.

In one example, the modified plurality of blocks of parameters are provided or loaded into the device configured to synthesize molecules, which then execute the cycles and process steps indicated by the modified plurality of blocks of parameters.

In one embodiment, each cycle 110 comprises a plurality of process steps PS1-PS4 and where each respective block 310 comprises a corresponding number of sub-blocks 320 of parameters as the number of process steps in the plurality of process steps PS1-PS4. The received user input is further indicative of one or more modifications of the sub-blocks. Controlling synthesizing further comprise applying the modified sub-blocks to control the process steps PS1-PS4 of the respective cycles.

In one non-limiting example, all cycles comprise a process step of “coupling”. Sub-blocks of parameters related to the “coupling” step for some blocks may then be modified. If sub-blocks of parameters related to the “coupling” step for all blocks are modified in the same way this is referred to as a global modification. If sub-blocks of parameters related to the “coupling” step for blocks of the same type are modified in the same way this is referred to as a modification of cycles of the same type, e.g., all cycles using the base “A”. If only selected sub-blocks of parameters related to the “coupling” step are modified, this is referred to as a modification of selected cycles.

In one embodiment, modifying the generated plurality of blocks comprises applying the user input globally to all cycles by modifying respective blocks of parameters.

In one embodiment, modifying the generated plurality of blocks comprises applying the user input to all cycles of the same type by modifying respective blocks of parameters.

In one embodiment, the user input is further indicative of selected cycles of the process description 300, where modifying the generated plurality of blocks comprises applying the user input to the selected cycles

In one embodiment, the target molecule is an oligonucleotide and the plurality of process steps PS1-PS4 at least includes detritylation 211, coupling 212, oxidation 213 and capping 214.

Additionally or alternatively, the respective sub-bloc for the process step detritylation 211 comprises parameter values for start level, end level, linear flow, efficiency limit, wash CV, wherein the respective sub-bloc for the process step coupling 212 comprises parameter values for base identity, Eq, Amidite concentration, coupling CT, percentage activator, push flow, recycle time and wash CV, wherein the respective sub-bloc for the process step oxidation 213 comprises parameter values for ox CT DNA, ox Eq DNA, ox CT RNA, ox Eq RNA, chase CV ox, wash CV ox, wherein the respective sub-bloc for the process step capping 214 comprises parameter values for cap CT, cap CV, chase CV ox and wash CV ox.

In one embodiment, the target molecule is an oligopeptide and the plurality of process steps at least includes detritylation, coupling and optionally capping.

In one embodiment, the method step 530 of receiving user input comprises rendering a representation of the synthesizing process description 300. The representation comprises representations of blocks 310 and/or sub-blocks 320 and/or parameter values 220, 440. Step 530 further comprises displaying the representation to a user. Step 530 further comprises receiving user input indicative of modification of the generated plurality of blocks of parameters by receiving user input indicative of interaction with the representation.

Additionally or alternatively, the interaction is at least indicative of modified parameter values and indicative of that the modified parameter values should be applied globally to all cycles by modifying respective blocks of parameters.

Additionally or alternatively, the interaction is at least indicative of modified parameter values and indicative of that the modified parameter values should be applied to all cycles of the same type by modifying respective blocks of parameters.

Additionally or alternatively, the interaction is at least indicative of selected cycles of the process description 300 and indicative of modified parameter values and indicative of that the modified parameter values should be applied to the selected cycles.

FIG. 6 illustrates a device 600 configured to synthesize molecules according to one or more embodiments of the present disclosure. The device 600 comprises a fluid network, e.g., comprising fluidly coupled pipes, fluidly coupled valves and fluidly coupled pumps.

The fluid network may be configured/controlled to receive fluids from fluid sources F1-F3 via a solvent valve VH9-IB. The fluid network may further be configured/controlled to direct the received fluids to a recirculation valve V9H-RC. The fluid network may further be configured to direct, via the recirculation valve V9H-RC, to a waste port, optionally via a waste valve V9H-01. The fluid network may further be configured to direct, via the recirculation valve V9H-RC, to a column valve V9H-C. The column valve V9H-C is configured to direct fluids to one or more columns 640 and/or receive fluids from the one or more columns 640. The fluid network may further be configured to direct, via the column valve V9H-C, fluids to a second solvent valve V9H-IA1. The fluid network may further be configured to direct, via the second solvent valve V9H-IA1, to fluid receptors, e.g., containers. The fluid network may further be configured to direct, via the second solvent valve V9H-IA1, fluids to a first amidite valve V9H-IA2 and or to a second amidite valve V9H-IA3. The first amidite valve V9H-IA2 may distribute amidites A21-A23 and the second amidite valve V9H-IA3 may distribute amidites A31-A33, e.g., distribute to containers.

Additionally or alternatively, the device further comprises a display 618.

Additionally or alternatively, the device further comprises an input device 617.

Additionally or alternatively, the device further comprises a control unit 610 comprising circuitry, the circuitry comprising processing circuitry and a memory, said memory comprising instructions executable by said processing circuitry, wherein the control unit 610 is communicatively coupled to each controllable part of the fluid network, e.g., pumps and valves. Wherein said device 600, comprising the control unit 610, is configured to perform any method steps described herein.

A selection of device components of the fluid network are communicatively coupled to the control unit 610 (shown as dashed lines), e.g., sensors and valves. Information on characteristics of the fluid network or characteristics of fluids carried by the fluid network may be measured by sensors and sent to the control unit 610 as control signals. The control unit 610 may react to the blocks of parameters and/or the information on characteristics of the fluid network and control valves in the fluid network by sending control signals.

In one embodiment, the device further comprises one or more sensors configured to measure characteristics of the fluid network. The control unit 610 is further communicatively coupled to each of the sensors. The sensors may be configured to measure pressure and/or measure pH and/or conductivity of the generated buffer solution. Any suitable sensor for performing measurements on fluids may be used.

In one example, the device is a OligoPilot II (also a trademark of the assignee of the present invention) and uses a flow-through design in which various conduits, pumps, and valves are constantly filled with liquid/fluids. Liquid introduced into a vessel (called a “column” in a flow-through device) displaces previously introduced liquid. This flow-through system is distinguished from a “batch” system in which liquids are introduced into a reaction vessel, the introduced liquids are flushed out, and the steps of introducing and flushing liquids is repeated. In such a batch device, the liquids are provided to the vessel by gas pressure and not with pumps. This approach can be used because a batch process has gaps in the flow of fluid.

In the OiigoPilot II device, first and second eight-way valves, each having eight individually selectable inlet ports, have output ports coupled to inlet ports of a first three-port valve of the type in which one and only one of the inlet ports must be kept open. Each of the two eight-way valves has four inlet ports coupled to receive one of four different types of amidites, and four inlet ports coupled to receive ACN (the flushing agent).

The outlet port of the first three-port valve is coupled to a first inlet port of a second three-port valve of the same type as the first. A second inlet port to this second three-port valve is coupled to a third eight-way valve that has various inputs including ACN, oxidizing agents, capping agents, and activator. The outlet port of the second three-way valve is provided to a valve that is coupled to an inlet side of a reagent pump for pumping liquid to the column through valve at the outlet side of the reagent pump. This last valve is also coupled to two pumps that are dedicated to pumping deprotectant and ACN at a higher flow rate than the reagent pump.

Liquids output from the column are provided through the valve at the pump's inlet side, to a monitor for detecting absorption of light to sense displaced trityl groups, and then to a waste valve that receives one input and has a number of separate outputs for waste. By selecting certain ports on the valves on the inlet and outlet sides of the reagent pump, the liquid can be circulated through the column for a desired time.

In the OligoPilot II machine, to introduce a next amidite into the column, one of the eight-way valves is set to receive a next amidite while another of the eight-way valves is set to receive the activator. The eight-way valves receiving the activator and the amidite are pulsed back and forth to introduce quantities of each alternatively.

To regulate the amounts of the liquids that are provided to the column, each of the pumps is initially calibrated. During operation, the pumps are activated a certain period of time to provide the desired quantities of liquid. Periodically, the pumps must be rechecked and recalibrated to avoid problems that can result from drifting in the pump. For the scale of synthesis involved, such flow calibration is sufficiently accurate for controlling the quantities of reagents delivered to the column. For larger scale synthesis, however, there is a need for more sophisticated means for controlling the delivery of the liquids.

In one embodiment, a device 600 is provided and is configured to synthesize molecules. The synthesizing comprises performing cycles of a plurality of process steps PS1-PS4, wherein the synthesizing is controlled by blocks of parameters. The device comprises:
a controllable fluid network,
an input device 617,
an optional display 618,
and a control unit 610 comprising circuitry, the circuitry comprising:
a processing circuitry, and
a memory, said memory comprising instructions executable by said processing circuitry, wherein the control unit 610 is communicatively coupled to controllable units of the fluid network, whereby said device is configured to perform the method described herein when the instructions are executed by said processing circuitry.

In one embodiment, a computer program is provided and comprises computer-executable instructions for causing the control unit 610, when the computer-executable instructions are executed on processing circuitry comprised in the control unit 610, to perform the method steps described herein.

The control unit 610 may be in the form of e.g., an Electronic Control Unit, a server, an on-board computer, a stationary computing device, a laptop computer, a tablet computer, a handheld computer, a wrist-worn computer, a smart watch, a smartphone, or a smart TV. The control unit 610 may comprise processing circuitry communicatively coupled to a transceiver configured for wired or wireless communication. The control unit may further comprise at least one optional antenna (not shown in figure). The antenna may be coupled to the transceiver and is configured to transmit and/or emit and/or receive wired or wireless signals in a communication network, such as WiFi, Bluetooth, 3G, 4G, 5G etc. In one example, the processing circuitry may be any of a selection of processing circuitry and/or a central processing unit and/or processor modules and/or multiple processors configured to cooperate with each-other. Further, the control unit 610 may further comprise a memory. The memory may e.g., comprise a selection of a hard RAM, disk drive, a floppy disk drive, a flash drive or other removable or fixed media drive or any other suitable memory known in the art. The memory may contain instructions executable by the processing circuitry to perform any of the steps or methods described herein. The processing circuitry may optionally be communicatively coupled to a selection of any of the transceiver the memory, one or more sensors, such as pH sensors, conductivity sensors and pressure sensors or any suitable type of sensor capable of measuring characteristics of the device and/or fluids processed by the device. The control unit 610 may be configured to send/receive control signals directly to any of the above mentioned units or to external nodes or to send/receive control signals via the wired and/or wireless communications network.

The wired/wireless transceiver and/or a wired/wireless communications network adapter may be configured to send and/or receive data values or parameters as a signal to or from the processing circuitry to or from other external nodes. E.g., measured pH or conductivity or generated volume of the buffer solution.

In an embodiment, the transceiver communicates directly to external nodes or via the wireless communications network.

In one or more embodiments the control unit 610 may further comprise an input device 617, configured to receive input or indications from a user and send a user input signal indicative of the user input or indications to the processing circuitry.

In one or more embodiments the control unit 610 may further comprise a display 618 configured to receive a display signal indicative of rendered objects, such as text or graphical user input objects, from the processing circuitry and to display the received signal as objects, such as text or graphical user input objects.

In one embodiment the display 618 is integrated with the user input device 617 and is configured to receive a display signal indicative of rendered objects, such as text or graphical user input objects, from the processing circuitry and to display the received signal as objects, such as text or graphical user input objects, and/or configured to receive input or indications from a user and send a user-input signal indicative of the user input or indications to the processing circuitry.

In a further embodiment, the control unit 610 may further comprise and/or be coupled to one or more additional sensors (not shown in the figure) configured to receive and/or obtain and/or measure physical properties pertaining to the device and/or or an atmosphere surrounding the device and send one or more sensor signals indicative of the physical properties of the device to the processing circuitry. E.g., a temperature sensor measuring ambient air temperature.

In one or more embodiments, the processing circuitry is further communicatively coupled to the input device and/or the display and/or the additional sensors and/or any of the units described herein.

In embodiments, the communications network communicate using wired or wireless communication techniques that may include at least one of a Local Area Network (LAN), Metropolitan Area Network (MAN), Global System for Mobile Network (GSM), Enhanced Data GSM Environment (EDGE), Universal Mobile Telecommunications System, Long term evolution, High Speed Downlink Packet Access (HSDPA), Wideband Code Division Multiple Access (W-CDMA), Code Division Multiple Access (CDMA), Time Division Multiple Access (TDMA), Bluetooth®, Zigbee®, Wi-Fi, Voice over Internet Protocol (VoIP), LTE Advanced, IEEE802.16m, WirelessMAN-Advanced, Evolved High-Speed Packet Access (HSPA+), 3GPP Long Term Evolution (LTE), Mobile WiMAX (IEEE 802.16e), Ultra Mobile Broadband (UMB) (formerly Evolution-Data Optimized (EV-DO) Rev. C), Fast Low-latency Access with Seamless Handoff Orthogonal Frequency Division Multiplexing (Flash-OFDM), High Capacity Spatial Division Multiple Access (iBurst®) and Mobile Broadband Wireless Access (MBWA) (IEEE 802.20) systems, High Performance Radio Metropolitan Area Network (HIPERMAN), Beam-Division Multiple Access (BDMA), World Interoperability for Microwave Access (W-MAX) and ultrasonic communication, etc., but is not limited thereto.

Moreover, it is realized by the skilled person that the control unit 610 may comprise the necessary communication capabilities in the form of e.g., functions, means, units, elements, etc., for performing the present solution. Examples of other such means, units, elements and functions are: processors, memory, buffers, control logic, encoders, decoders, rate matchers, de-rate matchers, mapping units, multipliers, decision units, selecting units, switches, interleavers, de-interleavers, modulators, demodulators, inputs, outputs, antennas, amplifiers, receiver units, transmitter units, DSPs, MSDs, TCM encoder, TCM decoder, power supply units, power feeders, communication interfaces, communication protocols, etc. which are suitably arranged together for performing the present solution.

Especially, the processing circuitry and/or processing means of the present disclosure may comprise one or more instances of processing circuitry, processor modules and multiple processors configured to cooperate with each-other, Central Processing Unit (CPU), a processing unit, a processing circuit, a processor, an Application Specific Integrated Circuit (ASIC), a microprocessor, a Field-Programmable Gate Array (FPGA) or other processing logic that may interpret and execute instructions. The expression “processing circuitry” and/or “processing means” may thus represent a processing circuitry comprising a plurality of processing circuits, such as, e.g., any, some, or all of the ones mentioned above. The processing means may further perform data processing functions for inputting, outputting, and processing of data comprising data buffering and device control functions, such as user interface control, or the like.

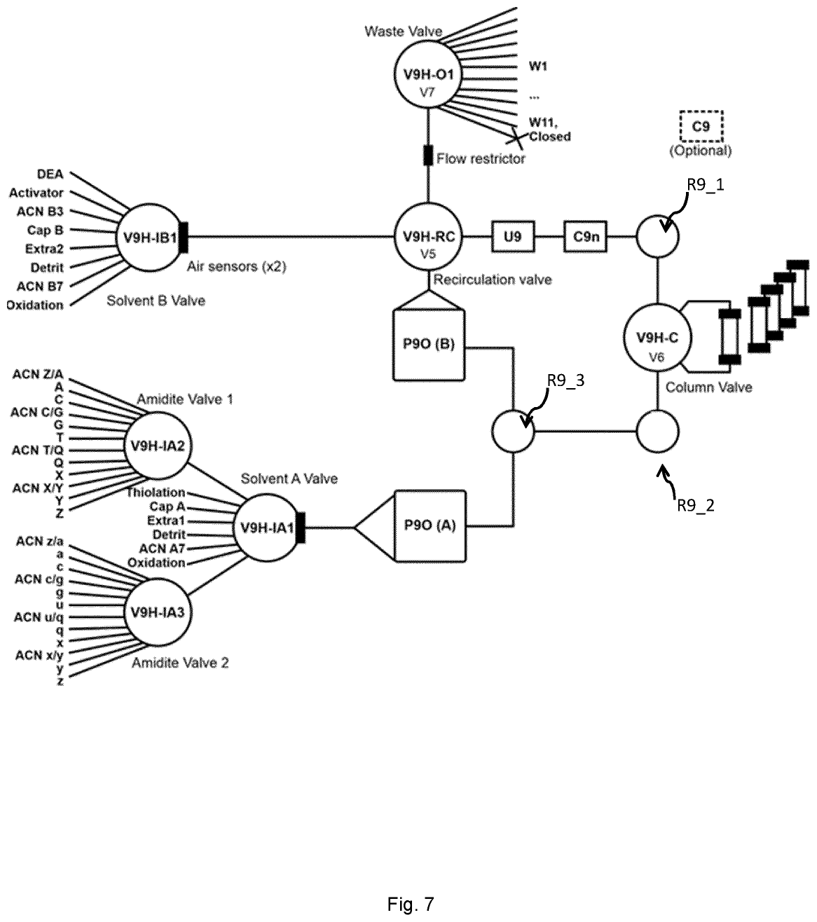

FIG. 7 illustrates an example of a device configured to synthesize oligonucleotides according to one or more embodiments of the present disclosure. The device is described with reference to FIG. 6. The device in FIG. 7 is fluidly coupled in a similar manner to what is described in FIG. 6. Optionally, an Ultraviolet sensor, UV sensor, U9 is fluidly coupled between the recirculation valve V9HRC and the column valve V9H-C. In addition, a conductance sensor C9n is further fluidly coupled between the recirculation valve V9HRC and the column valve V9H-C. Optionally, a first pressure sensor R9_1 is further fluidly coupled between the recirculation valve V9HRC and the column valve V9H-C. The UV sensor U9, the conductance sensor C9n and the first pressure sensor R9_1 are typically fluidly coupled in series, in any order suitable or practically selected.

Optionally, a second pressure sensor R9_2 is further fluidly coupled between the column valve V9H-C and the second solvent valve V9H-IA1. In addition, a third pressure sensor R9_3 is further optionally fluidly coupled between the second pressure sensor R9_2 and the second solvent valve V9H-IA1.

Optionally a pressure sensor P 90 (A) is fluidly coupled between the second or third pressure sensor R9_2, R9_3 and the second solvent valve V9H-IA1.

The second solvent valve V9H-IA1 is in this example provided with fluid outlet ports, e.g., for Thiolation, Cap A, Extra 1, Detrit, CAN A7, Oxididation. The second solvent valve V9H-IA1 is further fluidly coupled to a first amidite valve V9H-IA2 and/or to a second amidite valve V9H-IA3.

The first amidite valve V9H-IA2 is in this example provided with fluid outlet ports, e.g., for A, C, G, T, Q, X, Y, Z amidites, or any acetonitrile binding between them.

The second amidite valve V9H-IA3 is in this example provided with fluid outlet ports, e.g., for a, c, g, u, w, x, y, z amidites, or any acetonitrile binding between them.

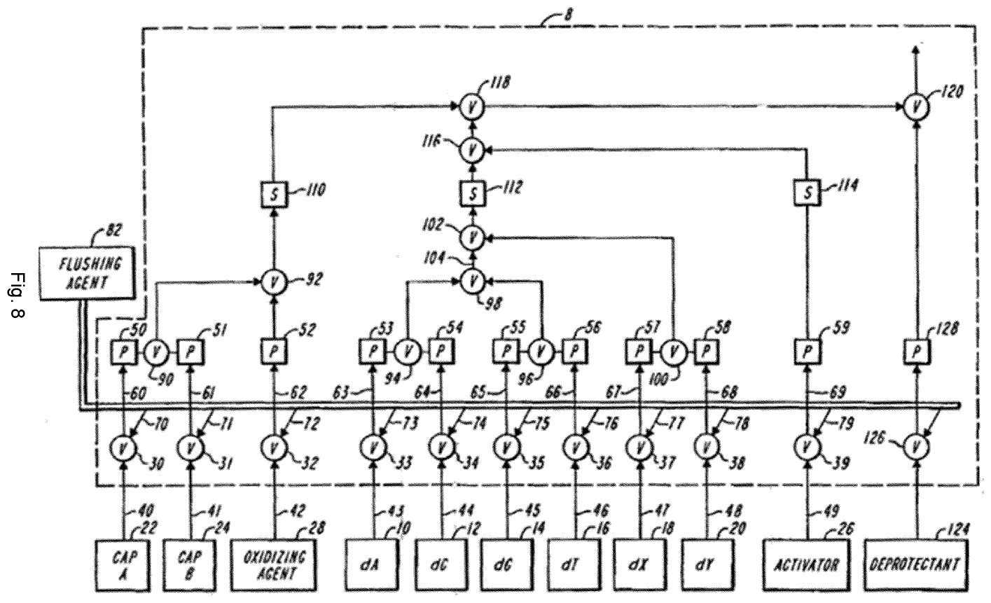

FIG. 8 illustrates an example of a device configured to synthesize oligonucleotides according to one or more embodiments of the present disclosure. In FIG. 8, the device 8 for synthesizing oligonucleotides has inlet ports for fluidly receiving liquid from a number of fluid sources/containers that hold different types of liquids, and an outlet port for providing selected liquids to a column. While described here in connection with a phosphoramidite method for synthesizing oligonucleotides, device 8 can readily be used with other methods for synthesizing oligonucleotides.

The four most commonly used amidites, which are the monomers used in the phosphoramidite method, are kept separately in containers 10, 12, 14, and 16, respectively. These amidites have a deoxyribose sugar, and therefore the amidites are deoxyadenosine, deoxycytidine, deoxyguanosine, and deoxythymidine, known as dA, dC, dG, and dT, respectively. Other amidites that may be used, referred to here as dX and dY, are kept in containers 18 and 20. All of the amidites are dissolved in ACN.

Other agents are also kept in separate containers. Capping agents, represented as Cap A and Cap B, are kept in containers 22 and 24; an activator, preferably tetrazole, is kept in container 26; an oxidizing agent is kept in container 28; and a deprotectant, preferably a detritylation agent, is in container 124. In the case of the oxidizing agent, two added valves and pumps can be used to select between two different types of oxidizing agents, one used for the formation of phosphodiester linkages, and the other for the formation of phosphorothioate linkages.

Each container 10-28 is fluidly coupled to a first optional inlet port of one of optional valves 30-39 (not in respective order) through respective conduits 40-49. Valves 30-39 are preferably three-port/three-way valves, i.e., each has three ports, typically one outlet port and two inlet ports, such that one, both, or neither of the inlet ports can be kept open. A second inlet port for each optional valve 30-39 is fluidly coupled through a respective conduit 70-79 to a conduit 80 that carries ACN from a flushing agent container 82. Each of valves 30-39 is fluidly coupled at its outlet port to a respective pump 50-59 through a conduit 60-69. Each of the liquids that is introduced is thus associated with an individual pumping module that includes a pump and a valve.

The modules are coupled downstream through a valving arrangement that is controlled to select liquids for introduction to the column. The outlet ports of pumps 50 and 51, which receive Cap A and Cap B, are fluidly coupled to the inlet ports of valve 90, the output port of which is fluidly coupled to a first inlet port of a valve 92. When capping agent is to be introduced, both inlet ports of valve 90 are typically kept open at the same time to combine the capping agents in equal amounts. The second inlet port of valve 92 receives an oxidizing agent from the output of pump 52. Accordingly, valve 92 can selectively provide one of a combination of Cap A and Cap B, an oxidizing agent, and ACN.

Pumps 53-58, which are fluidly coupled to receive dA, dC, dG, dT, dX, and dY, respectively, are paired together, and the outlets of these pumps are fluidly coupled to a part of the valving arrangement for selecting one of the amidites (or ACN) for introduction to the column. The outlet ports of pumps 53 and 54 are fluidly coupled to the inlet ports of a valve 94, and the outlet ports of pumps 55 and 56 are fluidly coupled to the inlet ports of a valve 96. The outlet ports of valves 94 and 96 are fluidly coupled to the inlet ports of a valve 98, which, in turn, provides ACN or one of amidites dA, dC, dG, and dT to a first inlet port of valve 102. Pumps 57 and 58, which selectively pump dX and dY are fluidly coupled to a valve 100, which provides at its outlet port a liquid to a second inlet port of valve 102. The outlet port of valve 102 thus provides one of the six amidites or ACN. Because one amidite is provided at one time, valves 94-102 generally have only one of the inlet ports open at one time.

Pump 59 for pumping an activator, preferably tetrazole, from container 26 is not paired with another pump.

The liquids output from valve 92, valve 102, and pump 59 are provided to a respective flow sensor 110, 112, and 114. These sensors are preferably each turbine flow meters, such as a Model FTO-3 produced by EG&G/Amtele AB, which have a rotating turbine for carefully metering a volume of fluid flow. The flow meters provide signals to a controller, which uses these signals to regulate pumps 50-59.

The amidite or ACN flowing through sensor 112 and the activator or ACN flowing through sensor 114 are provided to the inlet ports of valve 116. Because valve 116 is a three-way valve and can receive fluids at two inlet ports simultaneously, when an amidite is provided from valve 102 and activator is provided from sensor 114, these liquids can be mixed together within valve 116. The liquid flowing through sensor 110 is coupled to a first inlet port of valve 118, which receives at a second inlet port the liquid from the outlet port of valve 116. Only one of the inlet ports to valve 118 is typically open at one time if one is open at all.

The outlet port of valve 118 is fluidly coupled to a first inlet port of a valve 120. The second inlet port of valve 120 receives either ACN from container 82 or deprotectant from container 124. The ACN and the deprotectant are each provided to a pumping module that includes a valve 126 and a pump 128. The outlet port of pump 128 is fluidly coupled to the second inlet port of valve 120. Pump 128 is generally similar in design to pumps 50-59, but has a flow rate that is ten times greater than that of any of pumps 50-59.

The liquid from the outlet port of valve 120 is provided to a piezoelectric pressure transmitter 140, which senses the pressure in the liquid and provides to the controller an electrical signal that indicates the pressure of the flowing liquid.

The liquid is then provided to an ultrasonic air sensor 142 that has a sensor unit disposed in the conduit that carries the liquid, and a control unit coupled to the sensor unit. The air sensor continuously monitors the liquid to sense bubbles or gaps in the liquid in the conduit. Such bubbles or gaps should not occur in a flow-through system; rather, the various conduits should always have liquid in them. If bubbles are sensed, the flow of liquid can be shut off to prevent air from entering the column downstream, and an alarm is sounded.

The liquid is next provided to a UV monitor and transmitter 144. The monitor is preferably a Model UV-M/1, and the UV-transmitter is preferably a Model UV-P. Each of these models is distributed by the assignee of the present invention. The monitor has a light source for providing light, a filter for providing light at a first selected wavelength, and a light detector that senses an amount of UV absorption. The wavelength is selected so that UV absorption indicates the amount of nucleosides in the liquid. The monitor provides analog signals to the transmitter, which has high resolution analog/digital converters to convert the analog signals into digital information for use by a controller.

The liquid from UV monitor and transmitter 144 is then provided to a valve 146 which has a first outlet port 147 for coupling to a flow-through column 151 where the oligonucleotides are produced, and a second outlet port 148 that can be used to bypass column 151 during tests or during start-up when all the liquids conduits are being primed.

As indicated above, there are a number of steps in which liquids must be flushed out of column 151 (or at least sufficiently diluted). Consequently, provision is made to receive and monitor the liquids provided from these flushing processes. Column 151 is coupled to an inlet port of a valve 150, which receives the liquid displaced from column 151 when new liquid is introduced to the column. A second inlet port of valve 150 is coupled to outlet port 148 of value 146 for bypassing column 151 when desired.

The displaced liquid is provided at the outlet port of valve 150 to first and second UV monitors and transmitters 152, 154. These two are of the same general type as UV monitor and transmitter 144. Monitor and transmitter 152 uses the same wavelength as monitor and transmitter 144 to detect nucleosides displaced from column 151. By receiving signals from UV monitors and transmitters 144 and 152, the controller can determine the amount of nucleosides introduced into and displaced from column 151, and therefore can determine the difference that is left in the column due to reaction with the nascent oligonucleotide.

UV monitor and transmitter 154 uses a second selected wavelength (which could be in the visible range and not the ultraviolet), different from the first, for sensing the amount of DMT that is removed by the deprotectant. When the deprotectant is introduced and the amount of DMT sensed by monitor and transmitter 154 exceeds a first threshold, it indicates a start of a deprotectant cycle. When the sensed amount of DMT then falls below a second threshold, the controller determines that the deprotectant cycle is finished. While this process leaves an amount of deprotectant in the lines between the column and valve 120 (FIG. 1), this amount is small relative to the volume of the column. The sensing done by monitor and transmitter 154 could be performed with a conductivity sensor.

After the UV monitors and transmitters, the liquid passes through flow sensor 156. In addition to generally sensing displaced liquid flow, sensor 156 is also used to regulate the flow of pump 128. Sensor 156 is generally similar to sensors 110, 112, and 114, but preferably is a Model FT4-8, which accommodates a larger quantity of liquid flow.

From flow sensor 156, the liquid is provided to a back pressure valve, and then to a first waste valve 160. Valve 160 has a single inlet port and two outlet ports, the first of which is provided as a waste output, and the second of which is provided to a second waste valve 162. Valve 162 also has two outlet ports. Accordingly, the combination of valves 160 and 162 provide three separate outputs for waste that results from the flushing of the column. This separation is useful because the different waste liquids have different requirements for handling and disposal.

Each of the valves described above is preferably a pneumatically actuated chree-port/three-way diaphragm valve, preferably a model produced by Robolux AB, located in Lidingo, Sweden. The air valves for providing the pneumatic drive that controls the valves are controlled by solenoid valves that are coupled to the controller. Each of the pumps is preferably a vane pump with a magnetic coupling drive, produced by Castor, located in Italy. Most pumps are a Model MPA114AP, with a capacity of 3-48 liters per hour, and pump 128 is a Model MPA116AP with a capacity of 30-480 liters per hour.

Finally, it should be understood that the invention is not limited to the embodiments described above, but also relates to and incorporates all embodiments within the scope of the appended independent claims.

1. A computer implemented method performed by a device configured to synthesize molecules, wherein the synthesizing comprises performing cycles of a plurality of process steps (PS1-PS4), wherein the synthesizing is controlled by blocks of parameters, the method comprising:
receiving an indication of a target molecule,
generating a synthesizing process description (300) by generating a plurality of blocks (310) of parameters, each block (310) controlling process steps of a respective cycle,
receiving user input indicative of modification of the generated plurality of blocks of parameters,
modifying the generated plurality of blocks of parameters using the user input,
controlling synthesizing of the target molecule by applying the modified plurality of blocks of parameters to control of the respective cycles.

2. The method according to claim 1, wherein each cycle (110) comprises a plurality of process steps (PS1-PS4) and wherein each respective block (310) comprises a corresponding number of sub-blocks (320) of parameters as the number of process steps in the plurality of process steps (PS1-PS4),
wherein the received user input is further indicative of one or more modifications of the sub-blocks,
wherein controlling synthesizing further comprises applying the modified sub-blocks to control the process steps (PS1-PS4) of the respective cycles.

3. The method according to claim 1, wherein modifying the generated plurality of blocks comprises applying the user input globally to all cycles by modifying respective blocks of parameters.

4. The method according to claim 1, wherein modifying the generated plurality of blocks comprises applying the user input to all cycles of the same type by modifying respective blocks of parameters.

5. The method according to claim 1, wherein the user input is further indicative of selected cycles of the process description (300), wherein modifying the generated plurality of blocks comprises applying the user input to the selected cycles.

6. The method according to claim 1, wherein the target molecule is an oligonucleotide, and the plurality of process steps (PS1-PS4) at least includes detritylation (211), coupling (212), oxidation (213) and capping (214).

7. The method according to claim 6, wherein the respective sub-bloc for the process step detritylation (211) comprises parameter values for start level, end level, linear flow, efficiency limit, wash CV, wherein the respective sub-bloc for the process step coupling (212) comprises parameter values for base identity, Eq, Amidite concentration, coupling CT, percentage activator, push flow, recycle time and wash CV, wherein the respective sub-bloc for the process step oxidation (213) comprises parameter values for ox CT DNA, ox Eq DNA, ox CT RNA, ox Eq RNA, chase CV ox, wash CV ox, wherein the respective sub-bloc for the process step capping (214) comprises parameter values for cap CT, cap CV, chase CV ox and wash CV ox.

8. The method according claim 1, wherein the target molecule is an oligopeptide, and the plurality of process steps at least includes detritylation, coupling and optionally capping.

9. The method according claim 1, wherein receiving user input comprises:
rendering a representation of the synthesizing process description (300), wherein the representation comprises representations of blocks (310) and/or sub-blocks (320) and/or parameter values,
displaying the representation to a user,
receiving user input indicative of modification of the generated plurality of blocks of parameters by receiving user input indicative of interaction with the representation.

10. The method according to claim 9, wherein the interaction is at least indicative of modified parameter values and indicative of that the modified parameter values should be applied globally to all cycles by modifying respective blocks of parameters.

11. The method according to claim 9, wherein the interaction is at least indicative of modified parameter values and indicative of that the modified parameter values should be applied to all cycles of the same type by modifying respective blocks of parameters.

12. The method according to claim 9, wherein the interaction is at least indicative of selected cycles of the process description (300) and indicative of modified parameter values and indicative of that the modified parameter values should be applied to the selected cycles.

13. A device (600) configured to synthesize molecules, wherein the synthesizing comprises performing cycles of a plurality of process steps (PS1-PS4), wherein the synthesizing is controlled by blocks of parameters, the device comprising:
a controllable fluid network,
an input device (617),
a display (618),
and a control unit (610) comprising circuitry, the circuitry comprising:
a processing circuitry, and
a memory, said memory comprising instructions executable by said processing circuitry, wherein the control unit (610) is communicatively coupled to controllable units of the fluid network, whereby said device is configured to perform the method according to claim 1 when the instructions are executed by said processing circuitry.

14. A computer program comprising computer-executable instructions for causing a control unit (610), when the computer-executable instructions are executed on processing circuitry comprised in the control unit (610), to perform any of the method steps according to claim 1.

15. A computer program product comprising a computer-readable storage medium, the computer-readable storage medium having the computer program according to claim 14.
